(19) United States
(12) Patent Application Publication
(10) Pub. No.: US 2020/0188163 A1
(43) Pub. Date: Jun. 18, 2020

(54) THIXOTROPIC BINGHAM PLASTIC FLUID CONTRACEPTIVE METHOD

(71) Applicants: Joshua D. Levine, Carrboro, NC (US); Robert A. Levine, Guilford, CT (US)

(72) Inventors: Joshua D. Levine, Carrboro, NC (US); Robert A. Levine, Guilford, CT (US)

(21) Appl. No.: 16/607,880

(22) PCT Filed: Jun. 15, 2017
(86) PCT No.: PCT/US17/37694

§ 371 Date: Oct. 24, 2019

Related U.S. Application Data
(60) Provisional application No. 62/488,911, filed on Apr. 24, 2017.

(51) Int. Cl. A61F 6/06; A61K 9/00; A61K 47/38

(52) U.S. Cl. CPC A61F 6/06; A61K 47/38; A61K 9/0034

(57) ABSTRACT

A method of contraception for a female subject is provided, as well as a contraceptive product and a contraceptive product system. The method includes spraying an amount of a thixotropic contraceptive product that has Bingham fluid characteristics into a subject's vaginal vault, which contraceptive product comprises a viscosity increasing agent in the range of about 4-8% by weight and an aqueous carrier. The thixotropic contraceptive product is sprayed in a manner that forms a barrier configured to prevent spermatozoa from entering a cervix of the subject.

This patent application claims the benefit of U.S. Provisional Application No. 62/488,911, filed on Apr. 24, 2017, which provisional patent application is hereby incorporated by reference in its entirety.

### BACKGROUND OF THE INVENTION

### 1. Technical Field

The present invention relates to materials and methods for a contraceptive barrier, including applicators and method of applicator use.

### 2. Background Information

All barrier method forms of contraception currently available to women have significant disadvantages. Disadvantages using a diaphragm or cervical cap include: 1) the time, expense and personal discomfort in being fitted for the device; 2) the necessary planning involved in inserting and removing the device at the proper time; 3) the difficulty some women have in inserting the device; 4) the pain and general discomfort that some women experience when using the device; 5) the need for a high-degree of hygienic maintenance of the device, which may be difficult in locations lacking access to clean water; 6) the need to use the devices with spermicides, which often cause irritation and inflammation of the vaginal mucosa, and are associated with other health risks including an increased risk of HIV and other sexually transmitted infections (STIs); 7) the problem that the device may be pushed out of place during intercourse; 8) the need to be re-fitted for a new device after childbirth; and 9) the general messiness, and perceived unpleasantness of foam, suppository, cream and jelly spermicides.

Other methods of birth control such as hormonal manipulation by oral contraceptives and by injection or implant have well documented side-effects, including but not limited to vaginal bleeding, possible higher incidents of hormone dependent tumors, hypertension, thromboembolic disease, emotional changes, and migraines. Intrauterine devices (IUDs) are sometimes painful, need to be inserted by a healthcare professional and may predispose to uterine infection. Some or all of these factors may contribute toward making these contraceptive methods undesirable options for many women.

What is needed is a safe, hormone-free, nontoxic (preferably preservative-free), reliable form of birth control, that is easy and comfortable to use, and does not require a significant amount of planning and maintenance prior to and or after intercourse. The terms preservative, antimicrobial, microbicide are used interchangeably in the literature and sometimes omitted from lists of ingredients when the additive is used and listed as a fragrance or perfume or other constituent with antimicrobial properties.

### SUMMARY OF THE INVENTION

According to an aspect of the present disclosure, a method of contraception for a female subject is provided. The method includes spraying an amount of a thixotropic contraceptive product that has Bingham fluid characteristics into a subject's vaginal vault, which contraceptive product comprises a viscosity increasing agent in the range of about 4-8% by weight and an aqueous carrier. The thixotropic contraceptive product is sprayed in a manner that forms a barrier configured to prevent spermatozoa from entering a cervix of the subject.

According to any aspect or embodiment of the present disclosure, the step of spraying may include using an applicator that is configured to selectively introduce an amount of shear stress into at least a portion of the contraceptive product sufficient to cause liquefaction of that portion of contraceptive product.

According to any aspect or embodiment of the present disclosure, the step of spraying the amount of thixotropic contraceptive product includes spraying the contraceptive product in a manner such that the contraceptive product is deposited on the subject's ectocervix and vaginal mucosa.

According to any aspect or embodiment of the present disclosure, the step of spraying the amount of thixotropic contraceptive product may include a plurality of sprays of the contraceptive product, with each spray depositing a layer of the contraceptive product on the subject's ectocervix and vaginal mucosa, and each spray separated in time from a subsequent spray by a rest period, and the layers cumulatively forming the barrier.

According to another aspect of the present disclosure, a contraceptive product is provided that includes a viscosity increasing agent in the range of about 4-8% by weight, and an aqueous carrier.

According to another aspect of the present disclosure, a contraceptive product system is provided. The system includes a contraceptive product and an applicator. The contraceptive product includes a viscosity increasing agent in the range of about 4-8% by weight and an aqueous carrier. The applicator is configured to selectively introduce shear stress into at least a portion of the contraceptive product sufficient to cause liquefaction of that portion of contraceptive product.

According to another aspect of the present disclosure, a method of contraception for a female subject is provided. The method includes spraying an amount of a thixotropic contraceptive product that has Bingham fluid characteristics into a subject's vaginal vault, which contraceptive product comprises a viscosity agent and an aqueous carrier. The contraceptive product is configured to remain in a semi-solid state provided an amount of shear stress present within the contraceptive product is below a threshold shear stress amount, and transforms to a liquefied state if the amount of shear stress present within the contraceptive product equals or exceeds the threshold shear stress amount, and reverts to the semi-solid state upon the shear stress present within the contraceptive product dissipating below the threshold shear stress amount. The thixotropic contraceptive product is sprayed in a manner that forms a barrier configured to prevent spermatozoa from entering a cervix of the subject.

According to another aspect of the present disclosure, a method of treating one or more symptoms of vaginal dryness is provided. The method includes spraying an amount of a thixotropic product that has Bingham fluid characteristics into a subject's vaginal vault, which thixotropic product comprises a viscosity agent and an aqueous carrier. The thixotropic product that is substantially anti-microbial free, and is configured to remain in a semi-solid state provided an amount of shear stress present within the contraceptive product is below a threshold shear stress amount, and transforms to a liquefied state if the amount of shear stress present within the contraceptive product equals or exceeds the threshold shear stress amount, and reverts to the semi-solid state upon the shear stress present within the contraceptive product dissipating below the threshold shear stress amount.

According to another aspect of the present disclosure, a contraceptive product is provided that includes a viscosity agent and an aqueous carrier combined to form a thixotropic product. The thixotropic product is configured to remain in a semi-solid state provided an amount of shear stress present within the thixotropic product is below a threshold shear stress amount, and transforms to a liquefied state if the amount of shear stress present within the thixotropic product equals or exceeds the threshold shear stress amount, and reverts to the semi-solid state upon the shear stress present within the contraceptive product dissipating below the threshold shear stress amount.

According to another aspect of the present disclosure, a personal lubricant is provided that includes a viscosity agent and an aqueous carrier combined to form a thixotropic product. The thixotropic product is substantially antimicrobial-free, and is configured to remain in a semi-solid state provided an amount of shear stress present within the thixotropic product is below a threshold shear stress amount, and transforms to a liquefied state if the amount of shear stress present within the thixotropic product equals or exceeds the threshold shear stress amount, and reverts to the semi-solid state upon the shear stress present within the contraceptive product dissipating below the threshold shear stress amount.

According to any aspect or embodiment of the present disclosure, the viscosity agent may include a mixture of microcrystalline cellulose and sodium carboxymethylcellulose.

According to any aspect or embodiment of the present disclosure, the viscosity agent within the contraceptive product may be in the range of about 7.0% to about 8.0% by weight.

According to any aspect or embodiment of the present disclosure, the contraceptive product may be substantially preservative-free, or substantially antimicrobial-free, or both.

According to any aspect or embodiment of the present disclosure, the applicator may have a body with a reservoir configured to hold the contraceptive product, a distal end and a nozzle disposed at a distal end of the applicator.

According to any aspect or embodiment of the present disclosure, the applicator may have a reservoir that is a disposable cartridge.

According to any aspect or embodiment of the present disclosure, the applicator may be configured as a disposable.

According to any aspect or embodiment of the present disclosure, the applicator may be configured for multiple uses.

According to any aspect or embodiment of the present disclosure, the applicator may include a collapsible container sized to hold a single use amount of CP.

According to any aspect or embodiment of the present disclosure, the applicator may include a collapsible container sized to hold an amount of CP adequate for multiple uses.

According to any aspect or embodiment of the present disclosure, the contraceptive product may be configured to assume a semi-solid state while at rest, and to transform to a liquefied state upon introduction of an amount of shear stress into the contraceptive product, and to revert to the semi-solid upon dissipation of at least a portion of the amount of the shear stress, wherein during the reversion to the semi-solid state, the contraceptive product is configured to entrap spermatozoa.

According to any aspect or embodiment of the present disclosure, the contraceptive product barrier may be configured to be deficient in at least one element essential for microbial growth in order to inhibit microbial growth.

According to any aspect or embodiment of the present disclosure, contraceptive product may be deficient in at least one nutrient essential for microbial growth.

According to any aspect or embodiment of the present disclosure, the contraceptive product is deficient in an amount that allows for a decrease in one or both of preservatives or antimicrobials in the contraceptive product.

According to any aspect or embodiment of the present disclosure, the contraceptive product may be phosphate-free.

According to any aspect or embodiment of the present disclosure, the contraceptive product may be configured to remain in a semi-solid state provided an amount of shear stress present within the contraceptive product is below a threshold shear stress amount, and transforms to a liquefied state if the amount of shear stress present within the contraceptive product equals or exceeds the threshold shear stress amount, and reverts to the semi-solid state upon the shear stress present within the contraceptive product dissipating below the threshold shear stress amount.

According to any aspect or embodiment of the present disclosure, the threshold shear stress amount is produced by a force acting on the contraceptive product, which force is equal to or greater than one gravitational force.

Embodiments of the CP are also well suited for use as a personal lubricant. As will be described below, a CP having a viscosity agent in the concentration range of 4-8% by weight possesses characteristics well suited for use as a personal lubricant. As an example, a CP as described may be applied on the outside of the applicator to facilitate insertion of the applicator into the subject's vagina. A CP for use as a personal lubricant may be packaged in a spray container to enable the CP to be applied by spray application by either partner or both.

Personal lubricants are used by as much as 90% of sexually active couples engaging in various forms of sexual contact including vaginal and anal intercourse. Currently available lubricants include water-based lubricants, silicone-based lubricants, and oil-based lubricants. Water-based lubricants have the advantage of being non-disruptive to latex condoms, but their effectiveness during use may be limited because of drying; e.g., loss of water. Oil-based lubricants may be disruptive to condoms, can require cleaning agents for removal after use, and can stain clothing or fabric. Silicone-based lubricants do not evaporate nor do they degrade latex condoms, but they can degrade and interact with silicone based devices.

Currently available personal lubricants often contain elements (e.g., preservatives, antimicrobials, etc.) that can have deleterious side effects. Examples of such elements include petroleum-based products, synthetic preservatives referred to as “parabens”, phenoxyethanol, methyl polysiloxane, dimethicone, propylene glycol, glycerin, and chlorhexidine. Potential deleterious side effects include interference with the normal functions and permeability of a user's skin, various different forms of cancer, skin irritation, dermatitis, inflammation, interference with normal bacteria flora, monilial vaginitis, etc. The present CP does not include such elements and therefore avoids issues associated therewith.

Embodiments of the present CP can be used to treat symptoms of vaginal dryness generally resulting from postmenopausal atrophic vaginitis. Estrogen creams or systemic estrogens can be used to treat vaginal dryness, but may have significant side effects due to hormonal absorption. Hormone-free treatment products are available, but those products typically include one or more preservatives and an acrylic polymer. As indicated above, some preservatives may be irritants, and may alter normally occurring vaginal flora. The present disclosure treats the manifestation of vaginal dryness rather than the underlying cause, and avoids the aforesaid deleterious side effects. Embodiments of the present CP having a viscosity agent in the range of about 4-8% concentration by weight may be used as a treatment for atrophic vaginitis. These CP embodiments supply hydration to the vaginal mucosa and are sufficiently mucoadhesive. In most instances, an amount of CP (e.g., about 5-10 ml) applied periodically (e.g., daily, or every couple of days) would be adequate to treat symptoms of vaginal dryness. The CP can be applied by spray or by direct insertion of an applicator of gel since the formation of a cervical barrier is not needed when used as a personal lubricant.

### BRIEF DESCRIPTION OF THE DRAWINGS

FIG. 1 shows a Table I that contains results from experimental testing of embodiments of the present contraceptive product.

FIG. 2 shows four slides residing in a horizontal orientation with contraceptive product applied by spray to a surface of each respective slide.

FIG. 3 is a slide such as shown in FIG. 2 with a contraceptive product spray applied to a surface of a vertically oriented slide, which vertically oriented slide surface is aligned with a gravitational vector.

FIG. 4A is a diagrammatic view of a test tube, illustrating a CP composition that fell from the bottom of an inverted test tube.

FIG. 4B is a diagrammatic view of a test tube, illustrating a CP composition that remained in the bottom of an inverted test tube.

FIG. 5 shows a Table II that contains results from experimental testing of embodiments of the present contraceptive product.

FIG. 6 is a schematic diagram of an embodiment of a contraceptive product applicator.

FIG. 7 is a diagrammatic view of an embodiment of a contraceptive product applicator.

FIG. 8 is a diagrammatic view of an embodiment of a contraceptive product applicator, with mechanical advantage.

FIG. 9 is a diagrammatic view of an embodiment of a contraceptive product applicator.

FIG. 10A is a diagrammatic view of an embodiment of a contraceptive product applicator, shown in an at rest state.

FIG. 10B is a diagrammatic view of the applicator shown in FIG. 10A, with the piston of the fluid actuator collapsed.

### DETAILED DESCRIPTION

The present disclosure is directed to a contraceptive barrier method that utilizes a thixotropic composition as described herein after, and referred to as the contraceptive product (“CP”). Aspects of the present disclosure include an applicator that may be used with the present method.

The CP used within the present method possesses unique and functionally important rheologic and biologic properties. The CP may be defined as a composition that is a gel suspension that: a) resides within a semi-solid state when in a resting state (e.g., at room temperature or body temperature); b) can be transformed from the aforesaid semi-solid state into a liquid state upon the introduction of at least a sufficient amount of shear stress (i.e., a “yield shear stress amount”); and c) returns to the aforesaid semi-solid state in a relatively short period of time (e.g., less than 3 seconds) upon dissipation of the shear stress and re-assumption of the resting state. Because of these characteristics, the CP may be described as having characteristics like a Bingham plastic fluid and may be referred to as a pseudoplastic fluid. As a Bingham plastic fluid, a level of shear stress can be introduced into a portion of a CP, but the CP portion will not change state from semi-solid to liquid until a threshold level of shear stress (typically referred to as the “yield stress”) is present within the CP portion. The yield stress level will not be reached in most present CP embodiments, absent a purposeful agitation force, pumping force, or the like. In other words, forces equivalent to, or less than, gravity (e.g., “1 G”) acting on the mass of the CP will not produce sufficient shear stress within the CP to cause the CP to change state from a semi-solid to a liquid. For sake of clarity, the terms “gravity” or “1 G” as used herein refer to the constant gravitational attraction force “F” (i.e., F=mg, where m is the mass of the body and g is a constant vector with an average magnitude of 9.81 m/s² (32.2 ft/s²) on Earth) If a portion of the CP is subjected to a force adequate to create shear stress in the CP at or above the yield level, however (e.g., by a pumping action or other agitation), then the portion of the CP subjected to the sufficient shear stress changes to a flowable liquid state (i.e., the CP exhibits “shear thinning properties”). The ability of the CP to maintain a semi-solid state, to change to a liquid state upon introduction of sufficient shear stress, and to revert to a semi-solid state within a very short period of time is important for several reasons as will be described below.

Embodiments of the present disclosure CPs include one or more viscosity agents with an aqueous carrier. Examples of acceptable viscosity agents include, but are not limited to, microcrystalline cellulose, carboxyalkycellulose, sodium-carboxymethylcellulose, methyl cellulose, hydroxyethyl cellulose, hydroxypropyl cellulose, algin, carageenans, carbomers, galactomannans, polyethylene glycols, polyvinyl alcohol, polyvinylpyrrolidone, sodium carboxymethyl chitin, sodium carboxymethyl dextran, sodium carboxymethyl starch, xanthan gum, chitosans, mucilage (a naturally occurring plant constituent with a molecular weight of 200,000 or greater derived from botanicals such as acacia gum or gum Arabic), marshmallow, tragacanth, carrageen, guar, quince seed, psyllium, sterculia, comfrey, fenegreek, coltsfoot, Icelandic and Irish moss, flax or linseed, locust bean, coltsfoot, and slippery elm bark and the like. Numerous acceptable viscosity agents may be described as polymers due to their chemical composition. Combinations of any two or more of the foregoing may be used. In some embodiments, embodiments of the CP may also include rheology-modifying agents as described above.

Preliminary testing indicates that a blend of microcrystalline cellulose (“MC”) and sodium-carboxymethylcellulose (Na—CMC) performs particularly well as a viscosity agent within CP embodiments. A specific non-limiting example of a viscosity agent that can be used within a thixotropic CP according to the present disclosure is VIVAPUR® MCG produced by JRS Pharma LP of Patterson, N.Y., USA, and in particular VIVAPUR® MCG 811P which contains a blend of microcrystalline cellulose (“MC”) and sodium-carboxymethylcellulose (Na—CMC). The present disclosure is not limited to any particular aqueous carrier. In some embodiments, the aqueous carrier may be buffered saline, saline, distilled water, de-ionized water, or the like.

The present CP as described above is not limited to any particular embodiment. Specific CP formulations may vary depending on the particular application, and/or on the particular constituents disposed within the CP formulation. Preferably, the CP comprises elements that are generally recognized as safe (GRAS).

As described below, embodiments of the present CP exhibit muco-adhesiveness properties. The term “muco-adhesiveness” refers to the property of a substance to adhere to moist mucous membranes. The American Society of Testing and Materials has defined it as the state in which interfacial forces, which may consist of valence forces, interlocking action or hydrogen bonding or any combination of them, hold two surfaces together. The muco-adhesiveness of certain CP embodiments helps to prolong the residence time of an applied layer of the CP on a mucous membrane.

In some embodiments, a CP according to the present disclosure may be sterilized by heat or gamma radiation. Some limited reduction of viscosity may occur as a result of the sterilization, but may be addressed by increasing the concentration of the viscosity agent prior to heat or radiation.

Aspects of the present method include the use of a “CP applicator” configured to pert lit a user to selectively apply a coating of CP to a subject's ectocervix and vaginal mucosa in a manner and form that allows the coating to block entrance to the subject's cervical os. The CP applicator may be configured as a single use applicator or it may be configured for multiple uses.

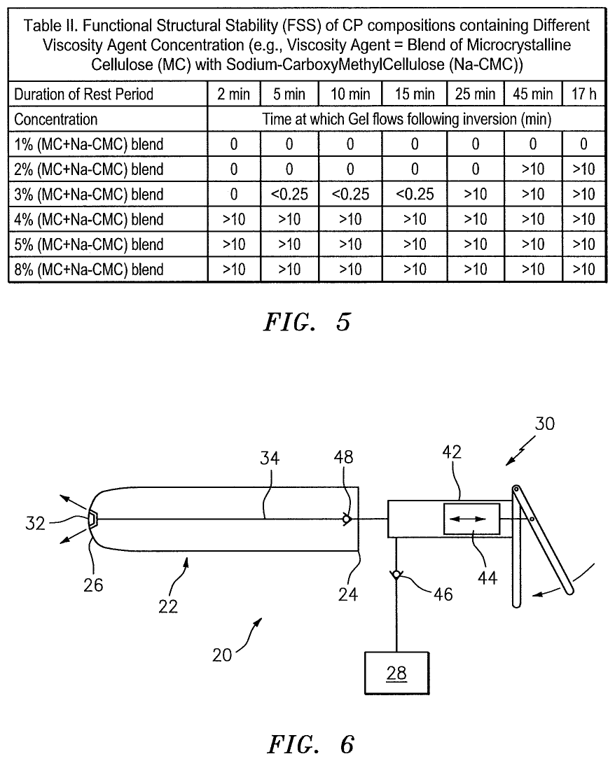

Referring to FIGS. 6-10B, embodiments of the CP applicator 20 typically include a tubular shaped body 22 having a proximal end 24 and a distal end 26. The distal end 26 and a portion of the body 22 are configured to facilitate insertion into the subject's vagina. The applicator 20 may be curved between the proximate and distal ends 24, 26 (e.g., see FIGS. 10A and 10B), or otherwise configured so that the distal end 26, when vaginally inserted, is disposable in close proximity to, but not abutting the subject's cervix, to facilitate delivery of CP to the target area. For example, an applicator that is configured to have a curvature of an erect human penis is an example of an acceptably shaped CP applicator. The present disclosure is not limited thereto, however. Embodiments of the present CP may assume any geometric configuration, including those that when rotated enable a spray application to a subject's ectocervix and vaginal mucosa, regardless of the specific orientation of the subject's cervix. The CP applicator 20 may be molded from silicon or similar materials, but is not limited to any particular material. The CP applicator is preferably plastically deformable (e.g., flexible), but sufficiently stiff to permit vaginal insertion. Some CP applicator embodiments may be substantially rigid.

The CP applicator 20 further includes a CP reservoir 28, a fluid actuator 30, and at least one nozzle 32. One or more conduits 34 fluidly connect the reservoir 28, the fluid actuator 30, and the at least one nozzle 32. The reservoir 28 is configured to hold an amount of CP, and is typically disposed at or near the proximal end 24. The CP reservoir 28 may be a compartment disposed within the applicator body 22 at or near the proximal end 24, or it may be a removable cartridge (or any type of container) that may be attached or disposed relative to the applicator 20; i.e., a disposable cartridge. The reservoir 28 may be sized to hold an amount of CP that is sufficient for more than one use (i.e., contain multiple doses of CP), or it may be sized to contain an amount of CP sufficient for a single use. As an example, in those embodiments wherein the reservoir 28 is a removable cartridge, the cartridge may be sized to contain a single use amount of CP after which use the cartridge may be removed and disposed of (or alternatively, the entire CP applicator 20 may be configured as a disposable containing a single use amount of CP). In some embodiments, the cartridge may be a pre-filled single use disposable. In some embodiments, the reservoir 28 may be a collapsible container sized to hold a single use amount of CP, or sized to hold an amount of CP adequate for multiple uses. In those embodiments wherein the reservoir 28 is a collapsible container (e.g., an aluminum tube), that collapsible container will allow the egress of CP, but will not allow any appreciable amount of air into the reservoir 28. The collapsible reservoir collapses because of air pressure as the CP is removed. In those CP applicator embodiments that include a collapsible container, the CP applicator may include a perforated tube that extends into the container to facilitate removal of the CP from the container.

The one or more nozzles 32 are disposed at the distal end 26, and each may be configured to produce a “spray pattern” of fluid ejected from the nozzle 32. A nozzle 32 configured to produce a conical-shaped spray pattern would be quite useful with the present disclosure. The present disclosure is not limited to any particular nozzle 32 configuration. As indicated above, the CP applicator may be configured for rotation about a rotational axis; e.g. a straight line extending through a center of the proximal end and into the body. In those embodiments wherein the CP applicator is curved, the nozzle may be pointed outwardly from the rotational axis, thereby creating a larger spray pattern when the applicator is rotated.

As indicated above, the fluid actuator 30 is in fluid communication with the CP reservoir 28 and the nozzle 32 located at the distal end 26 via one or more conduits 34. Any fluid actuator 30 that is operable to translate a useful amount of CP in a liquid form from the reservoir 28, through the conduit 34 to the distal end 26, and then eject the useful amount of CP at the distal end 26 may be used. Of course, the fluid applicator 30 is preferably able to create sufficient shear stress within a portion of the CP disposed within the reservoir 28 to cause that CP to be liquefied, which liquefied CP can then be translated to the nozzle 32. It is believed that a variety of different fluid actuator types may be used to translate CP in the manner described above, and the present disclosure is not therefore limited to any particular type. In some instances due to the semi-solid state of the CP in rest, it may be helpful to utilize a fluid actuator that provides mechanical advantage/leverage that facilitates the user's actuation of the fluid actuator.

A first example of a fluid actuator 30 is a pump spray type device (shown schematically in FIG. 6) that includes a pump having a housing 42 and a reciprocating piston 44 movable relative to the housing, a first one-way flow valve 46, and a second one-way flow valve 48. The first one-way flow valve 46 is in fluid communication with the pump housing 42 and the reservoir 28, and the second one-way flow valve 48 is in fluid communication with the pump housing 42 and the nozzle 32. In some instances, the nozzle 32 may be configured to act as both a nozzle and as a one-way flow valve. The reciprocating piston 44 may be described as having a “forward stroke” and a “return stroke”, which return stroke is opposite the forward stroke.

When the pump spray type fluid actuator 30 is operated, the piston 44 is translated within the pump spray housing 42 (referred to as a “forward stroke”) to apply force to the CP. The applied force is adequate to cause some of the CP to change state from semi-solid to liquid. During the forward stroke, the first one-way flow valve 46 is oriented to allow CP (e.g., in liquefied state) to be forced out of the pump spray housing 42, into the conduit 34, and thereafter to be expelled from the nozzle 32. Also during the forward stroke, the second one-way valve 48 is oriented to not permit fluid flow toward the reservoir 28. During the return stroke, the piston 44 is translated within the housing 42 in the opposite direction creating a negative pressure (suction) within the pump spray housing 42. The second one-way flow valve 48 is oriented to allow CP (e.g., in liquefied state) to be drawn out of the reservoir 28. The suction created during the return stroke is adequate to cause some amount of the CP to liquefy and be drawn into the pump spray housing 42. During the return stroke, the first one-way valve 46 is oriented to not permit fluid flow from the conduit 34 (or therefore the exterior) into the pump spray housing 42. A spring (not shown) may be used to create the force necessary to power the return stroke of the pump piston 44.

The type of pump spray device described above and shown schematically in FIG. 6 is also shown diagrammatically in FIGS. 10A and 10B. This pump spray type device that includes an internally disposed reciprocating pump, one or more one-way flow valves, and a reservoir (see FIG. 6). FIG. 10A shows the device in an “at-rest” configuration, and FIG. 10B shows the pump collapsed (i.e., the forward stroke has been completed). This type of linear reciprocal pump is known and used in some nasal spray applications.

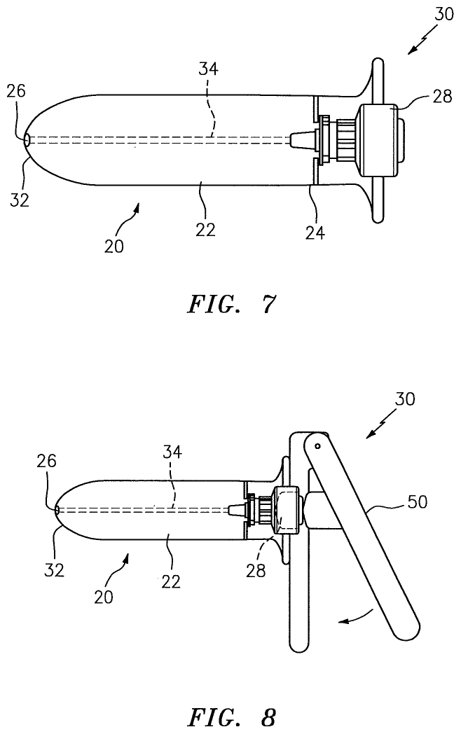

Now referring to FIGS. 7 and 8, a second example of a fluid actuator 30 is one wherein the reservoir 28 is configured such that it may be squeezed by the user to decrease the interior volume of the reservoir 28 thereby causing CP disposed within the reservoir 28 to be expelled (the specific mechanism in regards to how the thixotropic CP would be translated is described below). The reservoir 28 may be directly squeezed by the user, or it may be disposed within a portion of the CP applicator body 22 that can be squeezed (i.e., the applicator is squeezed, which in turn causes the reservoir to be squeezed), or it may be squeezed by a device 50 that utilizes mechanical advantage to facilitate the squeezing of the reservoir 28 (e.g., as shown in FIG. 8).

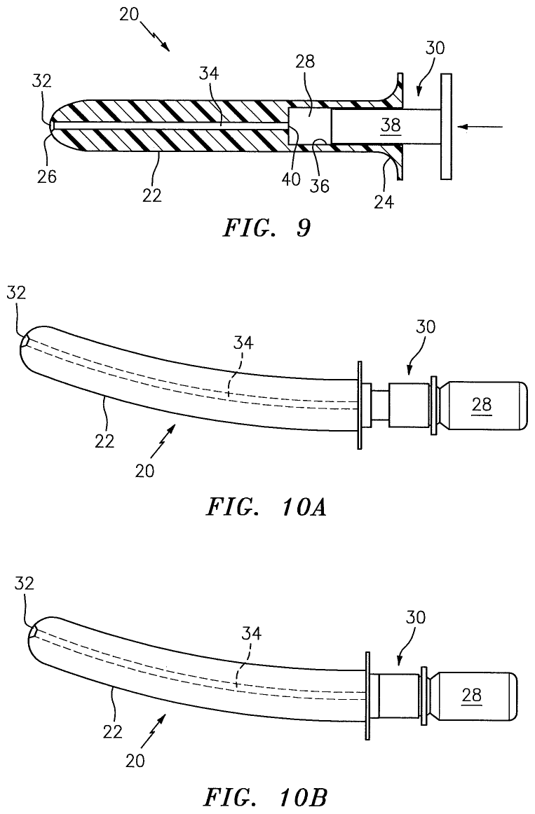

Now referring to FIG. 9, a third example of a fluid actuator 30 is a syringe type device (as shown in FIG. 9) that includes a barrel 36, a plunger 38 at least partially receivable within the barrel 36, and a port 40 in fluid communication with the barrel 36 and the conduit 34. The barrel 36 of the syringe acts as the reservoir 28, and the plunger 38 is receivable within the barrel 36 to selectively force CP out of the barrel 36, through the port 40, and into the conduit 34. This syringe type device may also be used with a device that provides mechanical advantage to apply force to the CP.

The present disclosure is not limited to any particular type of fluid actuator.

In some embodiments, the CP applicator 20 may include an indicator configured to indicate the volume of CP held within the applicator 20 and/or reservoir 28.

As an example of how the present contraceptive barrier method may be practiced, the user initially provides a CP applicator 20 and an amount of CP that is adequate to create a contraceptive barrier. As indicated above, the reservoir of the CP applicator 20 may be sized to contain an amount of CP adequate for a single use, or may be sized to contain an amount of CP adequate for multiple uses. The reservoir 28 is filled with an amount of CP at least adequate for a single use.

Prior to use, the user may express an amount of CP from the applicator once or twice before insertion so that the subject can be assured of sufficient CP remaining in the applicator and that the applicator is properly operating. The expressed CP may also be used as a personal lubricant to ease insertion of the applicator as well as to assure sufficient lubricity for coitus.

The CP applicator 20, “charged” with CP, is inserted into the subject's vagina a distance adequate to place the distal end of the applicator 20 in proximity to, but not in contact with, the user's ectocervix. The actuation of the CP applicator 20 applies a force to at least a portion of the CP disposed within the reservoir 28. The applied force is adequate to introduce a level of shear stress in the CP that is equal to or greater than the yield stress of the thixotropic CP which causes at least a portion of the CP to change from a semi-solid state to a liquid state so that it may be sprayed. To illustrate, consider a reciprocating piston type CP applicator as schematically shown in FIG. 6. The user applies a force to the CP by forcing a forward stroke of the piston. As a result of the applied force to the CP, at least a portion of the semi-solid state CP changes from semi-solid state to liquid state. The applied force is sufficient to force the liquid state CP out of the piston housing, through the conduit 34, and eject the liquid state CP from the nozzle(s) 32 located at the distal end 26 of the applicator 20. The one or more nozzles 32 preferably each to produce a “spray pattern” of fluid ejected from the nozzle; e.g., a conical-shaped spray pattern. Within a few seconds (or less) of a coating of CP being applied to the user's ectocervix and vaginal mucosa, the CP coating (deposited in liquid state) reverts back to a semi-solid state. The muco-adhesiveness properties of the CP facilitates adhesion of the CP to the mucous membranes of the subject's ectocervix and vagina.

The present method is not limited to any specific manner of spraying the CP. For example, the user may insert the CP applicator 20 until resistance to further insertion is felt, and then may withdraw it approximately a small amount so the distal end of the applicator is disposed a small distance from the user's ectocervix (e.g., ˜1.0 inch). The separation permits a wider spray pattern of liquid CP from the distal end of the applicator. It may also be useful to axially rotate the applicator during the spraying process to achieve a broader overall spray pattern (e.g., rotating a curved applicator like that shown in FIGS. 10A and 10B will result in a broader spray pattern).

Depending on the particular characteristics of the CP and the CP applicator, the coating produced by a single application of CP using the applicator may be sufficient to form a contraceptive barrier that will block the entrance to the subject's cervical os. However, in other instances it may be useful to repeat the above described deposition process a plurality of times. By depositing a plurality of CP coatings successively, one after the other, it is possible to collectively produce a substantially thicker CP coating; e.g., as each spray CP coating is deposited and begins to revert (or does revert) to semi-solid form, a subsequent spray CP coating is applied which adheres to the initial (now solidified) CP coating. In fact, testing indicates that it is possible to form a CP barrier having a thickness of several millimeters. As stated above, the thicker CP barrier will block the entrance to the subject's cervical os. As will be described below, there are distinct advantages associated with the ability of the present method to produce a relative thick CP layer adhered to the subject's ectocervix and vaginal mucosa. A distinct advantage of the present disclosure is that CP may be applied using a CP applicator without regard to the gravitational orientation of the user; e.g., the user may apply the CP while standing or laying down, etc.

It is believed that a deposition of about 10 to 20 mL will produce an adequate contraceptive barrier in most instances; e.g., a deposition that forms a CP barrier layer having a thickness that is several hundred micrometers (i.e., “microns”) to several millimeters thick on the subject's ectocervix and vaginal mucosa will perform adequately as a contraceptive barrier.

The ability of the present method to produce a liquid spray of CP provides several advantages not possible with existing contraceptive gels. For example, the ability of the present method to produce a contraceptive barrier by spraying CP in close proximity to the subject's ectocervix allows the user to create the aforesaid contraceptive barrier without regard to the gravitational orientation of the subject (and her cervix). The present method allows the CP barrier to be created regardless of whether the subject is standing upright or in a recumbent position; i.e., the applicator permits the CP to be sprayed onto the ectocervix and vaginal mucosa, the CP changes to a semi-solid state on the ectocervix and vaginal mucosa almost instantaneously, and the muco-adhesiveness of the CP keeps the CP barrier in place.

As indicated above, the ability of the CP to change state from a semi-solid to a liquid upon introduction of sufficient shear stress, and to revert to a semi-solid state within a very short period of time is important for several reasons. For example, the fact that the CP embodiments assume a semi-solid state while in a resting state negatively affects the ability of microbes to grow and replicate on or in the CP. This is characteristic of the CP is particularly beneficial while the CP is stored in a container for extended periods of time. The highly viscous CP (while in semi-solid state) acts as a diffusion barrier that inhibits the migration of microbes to nutrients that may be present within the CP, and vice versa. Once a microbe exhausts local nutrient sources and cannot travel to new nutrient sources, the ability of that microbe to replenish is significantly diminished. It is our belief that the small molecular size pores that exist within the semi-solid CP enhance the ability of the semi-solid CP to act as a diffusion barrier; e.g., a diffusion barrier to particulates as small as a virus or protein, and/or other relatively large molecules.

Another reason the present CP negatively affects the ability of microbes to grow and replicate on or in the CP, is because only a surface of the CP, which may be considered to be several microns thick and is a very limited amount of the total amount of CP, is exposed to air-borne microbes when the CP is stored for an extended period of time. As described above, the CP is a thixotropic composition, exhibiting Bingham fluid characteristics, including a yield stress level. Hence, an amount of CP disposed within a rigid reservoir will likely have only a single surface exposed to air while in the semi-solid state. The yield stress level of the CP is typically such that an applied force equivalent to, or less than, gravity (e.g., “1 G”) applied to the mass of the CP will not produce sufficient shear stress within the CP to cause the CP to change state from a semi-solid to a liquid. Hence, the exposed surface of the CP disposed within the reservoir does not change and only that exposed surface may be exposed to air-borne microbes. As indicated above, once a microbe on the exposed surface of the CP exhausts local nutrient sources and cannot travel to new nutrient sources (or vice versa), the ability of that microbe to replenish is significantly diminished.

The negative effect the CP has on the ability of microbes to grow and replicate on or in the CP may be enhanced by the CP being formulated to be deficient in one or more elements or chemicals essential for microbial growth (e.g., phosphorus, phosphates, or potassium), and/or formulated to be deficient in readily available chemicals or nutrients that support microbial growth, including metabolically available energy sources. Any one of these constituents, if absent (or extremely limited in quantity) will inhibit microbial growth. An example of an acceptable aqueous carrier of the CP preferably is a distilled and de-ionized water, made isotonic with sodium chloride, and then sterilized. The elimination of phosphates or any source of phosphorus containing compounds from the container or pumping mechanism will also inhibit microbiologic growth because phosphates are needed for DNA and RNA production. Present CP configurations may be completely phosphate free.

Another benefit associated with the present CP's thixotropic/Bingham qualities involves its ability to entrap spermatozoa. When the CP barrier is disposed on the subject's ectocervix, the semi-solid barrier will prevent spermatozoa from migrating through the barrier; e.g., the highly viscous CP (while in semi-solid state) acts as a diffusion barrier that inhibits spermatozoa migration there through to the cervical os of the subject. In addition, however, the nature of the CP is such that forces applied to the CP during coitus will very likely be sufficient to introduce shear stress into the CP at or above the yield stress point. As a result, the portions of the CP with shear stress at or above the yield stress will liquefy, but will quickly revert to semi-solid form once the shear stress is removed. Spermatozoa introduced into the subject's vagina will likely be mixed to some degree with the liquefied CP, and when the CP reverts back to a semi-solid form, the spermatozoa will be entrapped within the semi-solid CP. Spermatozoa residing within a vagina are known to have a lifespan of about two to three hours. Many, if not all, CP embodiments have the ability to remain in a semi-solid state for substantially longer than three hours (e.g., some CP embodiments tested remained in a semi-solid state longer than 24 hours). Thus, any spermatozoa trapped in semi-solid CP disposed within a subject's vagina, will be ensnared and therefore immobile for all of their useful life.

Still another benefit that is believed to be associated with the present CP's thixotropic/Bingham qualities involves its ability to decrease the potential for a transfer of sexually transmitted infections, including viral, bacterial, and protozoal infections. It is believed that the in vivo coating of the vaginal and cervical mucosa with the CP will decrease the possibility of a viral transmission (or other microbial transmission) from blood and seminal fluids from one sexual partner to the other, especially the male to female transfer of viral infection. The belief is based at least in part on the existing use of a semi-solid medium covering within plaque assays to prevent the virus infection from spreading indiscriminately. The lubricity of the CP will additionally help prevent female mucosal abrasion or male and female cutaneous abrasion, and thereby decrease both blood borne infections and infections with microbes that require their entry into the tissues of the body.

As can be seen from the above, the present CP and method differentiate themselves from other contraceptive gels and methods currently available in numerous ways. For example, the present method employs the CP in a manner that allows it to be used as an effective stand-alone contraceptive, one that does not need to be used with an additional barrier contraceptive device such as a diaphragm, a sponge, a cervical cap, or similar device to be effective. The present method may, however, be used in combination with other forms of birth control (i.e., condoms, fertility awareness methods, withdrawal, etc.) so as to increase contraceptive performance. In addition, the present method may be practiced along with chemical spermicides such as Nonoxynol-9, octoxynol-9, benzalkonium chloride, menfegol, and lactic acid; e.g., such spermicides may be added to the CP.

As indicated above, the present CP is a thixotropic composition that may be described as having characteristics like a Bingham plastic fluid. Thixotropy, as that term is used herein, is the property of certain gels or fluids that are viscous, semi-solid under at rest conditions, but become less viscous, flowable, liquid when subjected to a force (e.g., shaking, agitation, etc.) sufficient to introduce into the CP composition an amount of stress, and when the shear stress dissipates (after a period of rest, which is a function of the particular CP composition) return to the viscous, semi-solid state. A Bingham plastic is a viscous plastic material that behaves as a solid or semi-solid body at stresses below a threshold level, and only becomes flowable after being subjected to an amount of shear stress equal to or greater than the yield stress of that particular CP composition. The Bingham fluid resistance to flow at stress levels below the threshold level is time independent, which is also an important feature because, for example, positional changes of a stored amount of CP will not result in liquefaction/mixing of the CP which may facilitate microbial growth.

CP embodiments, as described herein, may be described as having non-Newtonian behavior with varying time dependent, shear stress dependent, and both time and force threshold dependent effects on their viscosity as well as time independent threshold yield stress thresholds. Because the present CP embodiments exhibit non-Newtonian fluid behaviors, the measurement of their viscosity is therefore both difficult to quantify due to their viscosity being shear stress and time dependent, and also dependent upon their undisturbed interval since their last sol-gel transformation.

To test the efficacy of the present method and CP, we conducted several tests to various CP compositions to evaluate their performance as a thixotropic composition, and to determine whether the same CP configurations exhibited characteristics consistent with a Bingham plastic. The tested CP compositions included those having viscosity agent concentrations by weight of 3%, 4%, 5%, 6%, 7%, 8%, 9%, and 10% in an aqueous carrier comprising a phosphate buffered saline. In these particular tests, the viscosity agent used was a microcrystalline cellulose and sodium-carboxymethylcellulose mixture. The specific viscosity agent used was a commercial product entitled VIVAPUR® MCG 811P, produced by JRS Pharma LP of Patterson, N.Y., USA.

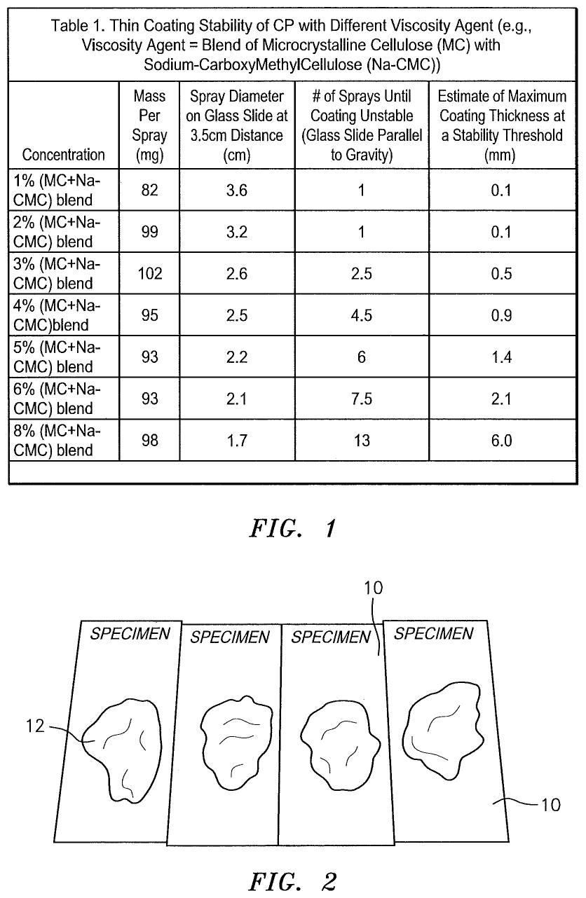

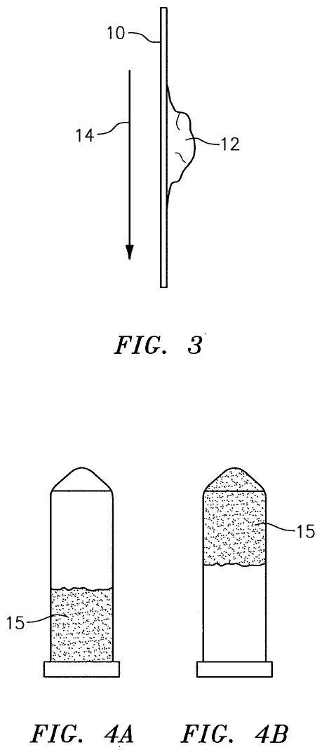

Now referring to FIGS. 1-3, a first series of tests included the following steps: (1) loading 15 mL of CP into a bottle with a digitally operated pump spray nozzle; (2) operating the pump to create a single spray directed at a horizontally oriented glass slide 10 (e.g., see FIG. 2), wherein the spray nozzle is located approximately 3.5 cm from the glass slide; (3) rotating the glass slide ninety degrees (90°) to a vertical position wherein the surface of the glass slide sprayed with the CP is now aligned with a gravitational vector 14 (e.g., see FIG. 3), and observing the slide 10 with the sprayed material coating 12 for a brief period of time (e.g., approximately five seconds) to observe the stability of the sprayed material coating relative to the glass slide 10; and (4) if the sprayed material coating remains stable, then steps 2 and 3 were repeated until the collective sprayed material coating became unstable; i.e., began to move relative to the glass slide 10. The experimental testing described above facilitated evaluation of CPs with different viscosity agent concentration by weight, and their ability to form a barrier layer and the limits of its thickness at 1 G As can be seen in FIG. 1, the estimated maximum CP coating thickness varied as a function of the viscosity agent concentration. The CP composition comprising 3% viscosity agent by weight produced an estimated maximum CP coating thickness of 0.5 mm (i.e., 500 micrometers or “microns”), and the CP composition comprising 8% viscosity agent by weight produced an estimated maximum CP coating thickness of 6.0 mm. The testing indicates that a stable barrier layer can be deposited on a mucosal tissue surface using a CP having a viscosity agent concentration in the range of about 3% to 10% by weight in saline. The present disclosure is not, however, limited to this range. Particularly useful CP layers were produced CP with a viscosity agent in the concentration range of between 4% and 8% by weight.

Now referring to FIGS. 4A, 4B, and 5, a second series of tests included the following steps: a) loading approximately 30 mL of a CP composition into a polypropylene tube, which tube had a conical base, had an approximately one inch (˜27 mm) diameter and an approximately four and one-half inches (˜114 mm) length; b) vigorously mixing each CP composition by hand to introduce shear stress into the respective CP; c) allowing the CP to rest within the tube for a given period of time; and d) subsequent to the rest period of time, inverting the tube to observe the respective tube for a period of time up to ten (10) minutes to determine if the CP moved within the tube as a result of gravity. The aforesaid testing was repeated utilizing rest periods (e.g., one to five minutes after liquefaction) shorter in duration than the 10-minute rest periods reflected in FIG. 5. The CP compositions used in the second series of tests was the same as those used in the first series of tests.

The testing indicated that several CP compositions remained in a semi-solid form and stable within the aforesaid test tubes when inverted upside down (i.e., rotated 180°) for twenty-four hours. In other words, certain of the CP compositions remained in a semi-solid form when subjected to one gravitational force (“1 G”) for over twenty-four hours. As can be seen in the data shown in Table II of FIG. 5, the CP compositions with 2% and 3% viscosity agent by weight did not form a semi-solid gel within the five-minute observation period. The CP composition with 4% viscosity agent by weight formed a semi-solid at 1.5 minutes, but did not adhere to the bottom of the tube when inverted; e.g., FIG. 4A diagrammatically depicts the CP 15 having fallen from the bottom of the inverted test tube. The CP composition with 5% viscosity agent by weight became semi-sold at five minutes, but after 30 seconds of inversion began slipping from the bottom of the inverted tube. The CP composition with 6% viscosity agent by weight became solid within less than one second. However, it began slipping within the tube within two minutes of the tube being inverted. The CP composition with 8% viscosity agent by weight solidified immediately after shaking and did not slip within the tube after being inverted for five minutes. The same 8% CP composition, when observed overnight, showed no signs of slipping after 48 hours of observation; e.g., FIG. 4B diagrammatically depicts the CP 15 having remained in the bottom of the inverted test tube. It should be noted, however, that these tests were preliminary in nature and involved CP compositions formulated with particular constituents, and alternatively formulated CPs may perform differently but may be configured to produce the same functionality.

The test results further indicated that a CP in a 4%-8% concentration range could be carried on one's person (e.g., in a container prior to use) and inverted and remain in place in a semi-solid form. The testing also indicated that CP compositions having a viscosity agent concentration by weight of less than 3% were likely not sufficiently viscous nor able to rapidly resume semi-solid state to be effective either as a contraceptive barrier. The test results also indicate that CP compositions having a viscosity agent in the range of 4-8% by weight will likely act as an effective inhibitor of microbial growth as described above. The ability of the CP compositions having a viscosity agent in the range of 4-8% by weight to change state from a semi-solid to a liquid, and then revert back to a semi-solid also suggests that the aforesaid CP compositions will also function to trap spermatozoa and thereby prevent the same from entering the subject's cervix.

Contraceptive gels and personal lubricants generally contain anti-microbial agents. These anti-microbial/chemical agents may kill or harm normal healthy bacterial flora in the subject's vagina and can cause overgrowth of harmful bacteria and yeast. The anti-microbial/chemical agents themselves may be irritants that cause burning or potentially are, or are feared to be, teratogens. The avoidance of potential or perceived teratogens is highly desirable, particularly contraceptive products since pregnancy still may occur. Since human testing for teratogenicity cannot be ethically performed, it is desirable to avoid contact with pharmacologic agents that cannot be tested in pregnant women and also desirable to avoid harsh chemicals that may cause irritation. A CP that is free of preservatives and chemicals including spermicides that may concern the patient is desirable, but not essential to the present disclosure, since a CP with a substantially reduced amount of preservatives is itself desirable.

The present CP, unlike existing contraceptive gels, which almost universally contain chemical spermicidal additives, functions because of its significantly high resting viscosity and thixotropic and Bingham fluid qualities, without the need for additional potentially harmful and irritating preservatives or antimicrobials or spermicides. As described above, the semi-solid, highly viscous form of present CP compositions negatively affects the ability of microbes to grow, and as a result the need for antimicrobials or preservatives is decreased or eliminated. Certain embodiments of the present CP provide an enhanced negative effect on the growth of microbes and therefore a decreased need for antimicrobials or preservatives; i.e., those CP compositions that have a decreased amount (or none) of chemicals and elements necessary to sustain microbial growth. For example, some CP embodiments may be configured to perform well using less than 50% less than the amount of antimicrobials and/or preservatives used in presently available contraceptive gels. In addition, some CP embodiments may be configured to be free of phosphorus or phosphorus containing compounds, which phosphorous is essential for DNA and RNA structure. The phosphate depletion is not effective in killing microbes, since they already contain the phosphate, but will inhibit the continued growth of any microbes that may be present.

Although this invention has been shown and described with respect to the detailed embodiments thereof, it will be understood by those skilled in the art that various changes in form and detail may be made without departing from the spirit and scope of the invention.

What is claimed:

1. A method of contraception for a female subject, comprising:
spraying an amount of a thixotropic contraceptive product that has Bingham fluid characteristics into a subject's vaginal vault, which contraceptive product comprises a viscosity agent in the range of about 4-8% by weight and an aqueous carrier;
wherein the thixotropic contraceptive product is sprayed in a manner that forms a barrier configured to prevent spermatozoa from entering a cervix of the subject.

2. The method of claim 1, wherein the viscosity agent includes a mixture of microcrystalline cellulose and sodium carboxymethylcellulose.

3. The method of claim 2, wherein the viscosity agent is in the range of about 7.0% to about 8.0% by weight in the contraceptive product.

4. The method of claim 1, wherein the contraceptive product is substantially preservative-free, or substantially antimicrobial-free, or both.

5. The method of claim 1, wherein the step of spraying includes using an applicator that is configured to selectively introduce an amount of shear stress into at least a portion of the contraceptive product sufficient to cause liquefaction of that portion of contraceptive product.

6. The method of claim 5, wherein the applicator has a body with a reservoir configured to hold the contraceptive product, a distal end and a nozzle disposed at a distal end of the applicator.

7. The method of claim 6, wherein the reservoir is a disposable cartridge.

8-11. (canceled)

12. The method of claim 1, wherein the step of spraying the amount of thixotropic contraceptive product includes spraying the contraceptive product in a manner such that the contraceptive product is deposited on the subject's ectocervix and vaginal mucosa.

13. The method of claim 12, wherein the step of spraying the amount of thixotropic contraceptive product includes a plurality of sprays of the contraceptive product, with each spray depositing a layer of the contraceptive product on the subject's ectocervix and vaginal mucosa, and each spray separated in time from a subsequent spray by a rest period, and the layers cumulatively forming the barrier.

14. The method of claim 1, wherein the contraceptive product is configured to assume a semi-solid state while at rest, and transforms to a liquefied state upon introduction of an amount of shear stress into the contraceptive product, and reverts to the semi-solid upon dissipation of at least a portion of the amount of the shear stress;
wherein during the reversion to the semi-solid state, the contraceptive product is configured to entrap spermatozoa.

15. (canceled)

16. The method of claim 1, wherein the contraceptive product is deficient in at least one nutrient essential for microbial growth.

17-18. (canceled)

19. The method of claim 1, wherein the contraceptive product is configured to remain in a semi-solid state provided an amount of shear stress present within the contraceptive product is below a threshold shear stress amount, and transforms to a liquefied state if the amount of shear stress present within the contraceptive product equals or exceeds the threshold shear stress amount, and reverts to the semi-solid state upon the shear stress present within the contraceptive product dissipating below the threshold shear stress amount.

20. (canceled)

21. A contraceptive product, comprising:
a viscosity increasing agent in the range of about 4-8% by weight; and
an aqueous carrier;
wherein the contraceptive product is substantially preservative-free, or substantially antimicrobial-free, or both.

22. The product of claim 21, wherein the viscosity agent includes a mixture of microcrystalline cellulose and sodium carboxymethylcellulose.

23. The product of claim 22, wherein the viscosity agent is in the range of about 7.0% to about 8.0% by weight in the contraceptive product.

24. The product of claim 21, wherein the contraceptive product is configured to remain in a semi-solid state provided an amount of shear stress present within the contraceptive product is below a threshold shear stress amount, and transforms to a liquefied state if the amount of shear stress present within the contraceptive product equal or exceeds the threshold shear stress amount, and reverts to the semi-solid state upon the shear stress present within the contraceptive product dissipating below the threshold shear stress amount.

25. The product of claim 24, wherein the threshold shear stress amount is produced by a force acting on the contraceptive product, which force is equal to or greater than one gravitational force.

26. The product of claim 24, wherein the contraceptive product is configured such that during the reversion to the semi-solid state, the contraceptive product is configured to entrap spermatozoa.

27-30. (canceled)

31. A contraceptive product system, comprising:
a contraceptive product having a viscosity increasing agent in the range of about 4-8% by weight, and an aqueous carrier; and
an applicator that is configured to selectively introduce shear stress into at least a portion of the contraceptive product sufficient to cause liquefaction of that portion of contraceptive product.

32-36. (canceled)

37. The system of claim 31, wherein the applicator has a tubular shaped body having a proximal end and a distal end, a fluid actuator, and at least one nozzle disposed proximate the distal end.

38. The system of claim 37, wherein the body is configured to facilitate vaginal insertion.

39. The system of claim 38, wherein the body is curved between the proximate end and the distal end and has a rotational axis;
wherein the nozzle is positioned such that when the applicator body is rotated about the rotational axis, the nozzle is directed outwardly.

40. The system of claim 38, wherein the applicator body has a length extending between the proximal and distal ends that is sufficient so that when vaginally inserted, the distal end can be positioned in close proximity to a cervix of a user.

41. The system of claim 37, wherein applicator includes a reservoir, and the fluid actuator is in fluid communication with the reservoir and the nozzle via one or more conduits.

42. The system of claim 41, wherein the fluid actuator is the element of the applicator configured to selectively introduce shear stress into the at least a portion of the contraceptive product sufficient to cause liquefaction of that portion of contraceptive product, and the fluid actuator is configured to selectively translate a useful amount of the liquid contraceptive product from the reservoir, through the one or more conduits to the distal end, and then eject the useful amount of contraceptive product from the nozzle.

43. The system of claim 41, wherein the fluid actuator is a pump spray type device that includes a pump having a housing and a reciprocating piston movable relative to the housing, a first one-way flow valve, and a second one-way flow valve.

44-57. (canceled)
